(19) United States
(12) Patent Application Publication
(10) Pub. No.: US 2025/0364094 A1
(43) Pub. Date: Nov. 27, 2025

(54) SYSTEMS AND METHODS FOR AUTOMATICALLY GENERATING A RECORD IN A DIGITAL COMMUNICATION NETWORK

(71) Applicant: Sequelae, Inc., Minneapolis, MN (US)

(72) Inventors: Mark Briesacher, Park City, UT (US); James LaBelle, Encinitas, CA (US); Benjamin W. Ehlert, Palo Alto, CA (US); Sara Taylor, Edina, MN (US); Ryan Michael Rodeback, Edina, MN (US)

(73) Assignee: Sequelae, Inc., Minneapolis, MN (US)

(21) Appl. No.: 19/210,707

(22) Filed: May 16, 2025

Related U.S. Application Data
(60) Provisional application No. 63/650,512, filed on May 22, 2024.

(51) Int. Cl. G16H 10/60; G16H 10/20

(52) U.S. Cl. CPC G16H 10/60; G16H 10/20

(57) ABSTRACT

Systems and methods for automatically generating a record in a digital communication network (DCN) are provided. A DCN is provided for a plurality of members of a population to communicate with each other and/or with the DCN. At least one communication is analyzed and a record is generated for an individual member based on the analysis of the at least one communication. The record includes one or more lifestyle activities engaged in by the individual member, biometric data reported to the DCN regarding the individual member, and/or information regarding one or more relationships of the individual member. The record is also provided to the individual member through the DCN.

### CROSS REFERENCE TO RELATED APPLICATIONS

This application claims the benefit of U.S. Provisional Application No. 63/650,512, filed on May 22, 2024, which application is incorporated herein by reference in its entirety.

### BACKGROUND

Various embodiments relate generally to health care systems, methods, devices and computer programs and, more specifically, relate to automatically generating a record or journal based on interactions in a digital communication network.

This section is intended to provide a background or context. The description may include concepts that may be pursued, but have not necessarily been previously conceived or pursued. Unless indicated otherwise, what is described in this section is not deemed prior art to the description and claims and is not admitted to be prior art by inclusion in this section.

Computers have changed the way people interact. Digital communication networks, which can include social media where individuals can interact with others online, connect many with a community. The online community can be utilized to help build positive behaviors and encourage people to make improvements in their lives. Additionally, computers have enabled new ways to get people to view their situations.

What is needed is a way to generate a record or journal for an individual member of a population based on interactions in a digital communication network.

### Record

Example aspects of the present disclosure include:

A method for improving communications between an individual member and a healthcare provider according to at least one embodiment of the present disclosure comprises providing a digital communication network (DCN) for a plurality of members of a population to communicate with each other; analyzing at least one communication in the DCN; generating a record for the individual member in the DCN based on the analysis of the at least one communication, the record including at least one of: one or more lifestyle activities engaged in by the individual member, biometric data reported to the DCN regarding the individual member, and information regarding one or more relationships of the individual member; and providing the record to the individual member through the DCN.

Any of the aspects herein, wherein the record comprises a section having one or more prepared questions the individual member can ask the healthcare provider, and wherein the one or more prepared questions include questions regarding medical care for the individual member.

Any of the aspects herein, wherein the questions are prepared automatically through the analysis of the at least one communication.

Any of the aspects herein, wherein the record is generated prior to an expected interaction with the healthcare provider by the individual member.

Any of the aspects herein, further comprising determining, by the DCN, the expected interaction based on the analysis of the at least one communication by the individual member.

Any of the aspects herein, wherein the record is prepared in at least one of a written format and a digital format.

Any of the aspects herein, further comprising: generating a link to the record; and providing the link to the healthcare provider.

Any of the aspects herein, wherein the lifestyle activities are prepared in at least one of a narrative form, a first person perspective, and a list form.

Any of the aspects herein, wherein the list form comprises at least one section of new activities, activities continued, activities dropped, and activities contemplated.

Any of the aspects herein, wherein the biometric data is derived in part from at least one of biometric data and psychometric data submitted to the DCN by the individual member.

Any of the aspects herein, wherein at least one of the biometric data and the psychometric data is derived from a biometric sample.

Any of the aspects herein, wherein the biometric sample is obtained from at least one of a home test, a wearable device, a psychometric instrument, a psychosocial instrument, and a psychological instrument.

Any of the aspects herein, wherein the wearable device comprises at least one of an accelerometer, a HR monitor, a HRV monitor, a continuous glucose monitor, a continuous ketone monitor, a skin temperature monitor.

Any of the aspects herein, wherein the biometric sample comprises at least one of a saliva sample, a blood sample, a breath sample and a stool sample.

Any of the aspects herein, further comprising providing the home test through the DCN.

Any of the aspects herein, wherein the record further includes analytics based on a combination of a plurality of biometric readings and changes in the biometric readings over time.

Any of the aspects herein, wherein the information regarding relationships is determined by analyzing at least one of contents of posts in the DCN and the at least one communications in the DCN.

Any of the aspects herein, wherein the method is used to at least one of: manage health care cost of the population; reduce health care risk in the population; and slow progression of an adverse health condition.

A method for improving communications between an individual member and a healthcare provider according to at least one embodiment of the present disclosure comprises providing a digital communication network (DCN) for a plurality of members of a population to communicate with each other; analyzing at least one communication in the DCN; generating a record for the individual member in the DCN, the record including at least one of: one or more lifestyle activities engaged in by the individual member, biometric data reported to the DCN regarding the individual member, information regarding one or more relationships of the individual member, and one or more prepared questions the individual member can ask the healthcare provider; determining an expected interaction between the individual member and the healthcare provided based on the analysis; and providing the record prior to the expected interaction.

A system to improve communications between an individual member and a healthcare provider according to at least one embodiment of the present disclosure comprises a computer processor; a data repository in communication with the computer processor and storing: at least one communication, and a record having at least one of: one or more lifestyle activities engaged in by the individual member, biometric data reported to the DCN regarding the individual member, and information regarding one or more relationships of the individual member a communication analyzer which, when executed by the computer processor, analyzes the at least one communication; a digital communications network which, when executed by the computer processor, provides a network for members of a population to interact with each other; and a server controller which, when executed by the computer processor: generate the record for the individual member based on the analysis of the at least one communication; and provide the record to the individual member.

### BRIEF DESCRIPTION OF THE DRAWINGS

Aspects of the described embodiments are more evident in the following description, when read in conjunction with the attached Figures.

FIG. 1 shows a simplified block diagram of devices, in accordance with one or more embodiments.

FIG. 2 is a logic flow diagram that illustrates the operation of a method, in accordance with one or more embodiments.

FIG. 3A shows an example of a computing system, in accordance with one or more embodiments.

FIG. 3B shows an example of a network, in accordance with one or more embodiments.

### DETAILED DESCRIPTION

Various embodiments are directed to automatically generating a record of interactions and/or communications in a DCN by an individual member. Such record may also be referred to as a journal, a health journal, and/or a summary. Automatic journaling and/or recording provides a means for an individual member to communicate their personal health journey. In turn, the individual member can share their journey with the DCN, the rest of the healthcare system, and/or their primary care physician. The record and/or journal can be automatically generated from, for example, the individual member's interactions with the DCN, including posts, experiences, biometric results, discussions and the like. Recording and/or journaling may be triggered for production when, for example, the individual member indicates (directly or indirectly) that they have an upcoming interaction with the healthcare system. The record and/or journal may also include questions for the individual to ask their physician.

By way of background, members of a population live their lives in many communities in the real world and online. Often a healthcare system does not take into account these communities and the social interactions between members within these communities. The individual members themselves may not realize how much influence these communities have on their healthcare (both physical and mental). Automatic recording and/or journaling of various communications and interactions within these communities can bring the importance of these real-world health modulators into the healthcare system in a low friction way.

Digital health applications typically provide members access to data about their health or health-related actions and often allow them to record observations about how they feel. This can assist in recording and/or journaling but in a minimal way.

Automatic recording and/or journaling according to embodiments of the present disclosure may be used to better record and present information about the individual member. For example, the individual member's information may aggregated, rearranged, and/or summarized and provided to caregivers and/or physicians in order to better evaluate the individual member.

This may be based, in part, on creating greater awareness of lifestyle and medial situations to the individual member. A lifestyle (or behavior) can be evaluated based on the individual member's information. Such recorded information may be used to help identify the individual member's condition, for example, to evaluate when a health change occurred.

Additionally, the individual member's information can be used to provide the individual member a social aspect. As a social process, value can be placed on those members of a community that have relevant conditions. The recording and/or journaling from the members can be provided in order to help spread knowledge about the aspects of an individual member's health journey that may be more instructional to the community as a whole. This can help increase the number of members of the community thinking about an action, for example, creating an atmosphere where people can learn about others who have undergone similar experiences.

Additionally, the community can be used to help support the individual member in other ways. Lifestyle interventions are inherently safe. They use the philosophy that any action now is preferred to a “better” action later. They also support the concept that ideas and communication are healthcare. The community can be used to encourage such lifestyle interventions. Such community may use the philosophy that any action now is preferred to a “better” action later and also support the concept that ideas and communication are healthcare. Conventionally, lifestyle change has been looked at as in individual pursuit, such as plans personalized just for the individual. Further, medicine is typically a one-on-one activity (reinforced by the privacy concepts the system is based on). In the present disclosure, lifestyle change is seen as highly driven by social parameters and the impact on social parameters is critical.

Conventional lifestyle applications may tell individuals the “right thing to do”, which could be right, but given the complexity of the lifestyle change, is likely not to occur. Often, if the lifestyle change suggested works, they can make the individual more dependent on things outside the individual's control. On the other hand, communities share experiences, not expertise, which individuals can try and if they work for them, is a success. In some cases, success can range from slowing the progression of adverse conditions to managing a disease, or the overall risk level in a population.

Lifestyle change may be supported by communities that provide support, ideas, and, in the case of these ideas, access to tools to provide objective data to make meaningful lifestyle changes in an individual member or members of a population. For example, in a community where a person is a peer, the actions they take and learn from are that their volition and may result in increased agency (or autonomy) or self-efficacy. This not only increases the chances of continuous lifestyle improvement, but improved outcomes throughout the heath system.

Further, online communities such as the DCN can be provided so that people can learn about healthy lifestyle practices and work to improve their health between clinical touchpoints, such as office visits. To help incentivize healthy behaviors, individuals can use tools like in-home tests and biosensors that measure how well their health actions are working.

Thus, it is desirable to provide systems and methods that can automatically generate records and/or journals to track and/or present information about an individual member's health journey based on the individual member's interactions with in a DCN. Such records can beneficially provide insight into the individual member's life and lifestyle to, for example, a healthcare provided.

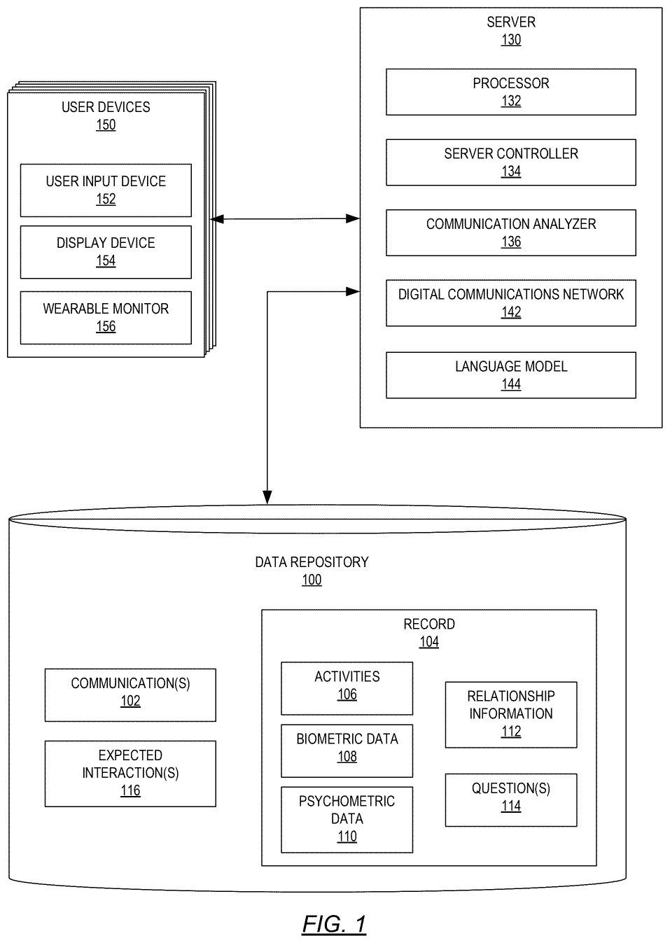

The system shown in FIG. 1 includes a data repository (100). The data repository (100) is a type of storage unit or device (e.g., a file system, database, data structure, or any other storage mechanism) for storing data (described below). The data repository (100) may include multiple different, potentially heterogeneous, storage units and/or devices.

The data repository (100) stores communications (102). The communications (102) are communications from members in a digital communication network (DCN) (142) (described in more detail below). The communications (102) may be, for example, text-based, image-based, multimedia communications, audio communications, or any combination thereof. The communications (102) can also be posts in the DCN (142) such as posts in an online forum and/or social media page. The communications may be between members, communications in a forum, communications with the DCN (142), or communications generated by the DCN (142) (e.g., communications from a conversation bot, also referred to as a chat bot).

The data repository (100) also stores a record (104). The record (104) is a record (104), journal, or summary of an individual member's communications (102) and/or interactions with other members of the population and/or the DCN (142) itself. The record (104) can also include information about the individual member provided by the individual member to the DCN (142). The record (104) can be prepared in either a written format and/or a digital format. The record (104), which can also be referred to as automatically journaling, can allow for automatic recordation and presentation of various health conditions and events of the individual member. Such automatic recording can be provided without increasing the cost of a healthcare system. Such recording can also be used to improve an individual member's awareness of their condition. For example, the record (104) can show a progression or regression of a disease to the individual member. The record (104) can include various information and sections that are useful to the individual member and/or the individual member's healthcare provider, as described below.

The record (104) includes a section having one or more lifestyle activities (106) engaged in by the individual member. The one or more lifestyle activities (106) includes information about the types of activities that the individual member may have participated in, is participating in, or is contemplating participating in. The lifestyle activities (106) may be prepared in a narrative form, a first-person perspective, and/or a list form. In embodiments where the lifestyle activities (106) are prepared in a list form, the list form may include at least one section of new activities, activities continued, activities dropped, and/or activities contemplated.

The record (104) can also include biometric data (108). The biometric data (108) may be derived from, for example, a biometric tool, an at home test, a wearable device, a biometric sample, answers to a survey, and/or a psychometric instrument. The at home test may be provided to the individual member by, for example, the DCN (142) or instructions to obtain the at home test may be provided to the individual member. When the biometric data (108) is a biometric sample, the biometric sample may be, for example, a saliva sample, a blood sample, a breath sample and/or a stool sample.

Biometric data (108) can be used in many instances such as the provisioning and commissioning of healthcare. For example, biometric data (108) can be used to diagnose diabetes, prescribe hypertension medication when a patient's blood pressure exceeds a certain value, prescribe cancer drugs, etc. In another example, cancer drug can be available for reimbursement if a certain genetic signature is present in the tumor. The decision tree and other logic behind these relationships considers many things including what is known about the variance in the intervention's efficacy, therapeutic index, the accuracy and precision in the biometric data (108)'s measurement, as well as the cost-benefit ratio of the intervention compared with other interventional options.

The record (104) can also include psychometric data (110). The psychometric data (110) is data derived from the individual member and can be used to forecast or predict the individual member's behavior. The psychometric data (110) may also be derived from, for example, a wearable device, a biometric sample, answers to a survey, a psychometric instrument, a psychosocial instrument, a psychometric instrument, and/or ecological momentary assessment (EMA) derived data.

The biometric data (108) and the psychometric data (110) can also be used in a “feedback” decision tree where, for example, an intervention or treatment can be prescribed or recommended to an individual member. Then the biometric data (108) and the psychometric data (110) can be obtained to study the effects or results of the intervention or treatment. More specifically, the record (104) can include analytics based on the biometric data (108) and/or the psychometric data (110) including biometric and/or psychometric readings and/or changes in the biometric and/or psychometric readings over time.

The record (104) can also include relationship information (112). The relationship information (112) may include relationships between the individual member and other members of the DCN (142). The relationship information (112) can be derived from analyzing the communications (102) including, for example, contents of posts in the DCN (142) and/or communications (102) between the individual member and the other members of the DCN (142).

The record (104) can also include question(s) (114). The questions (114) include one or more prepared questions that the individual member can ask their healthcare provider. The questions (114) may include, for example, questions regarding medical care for the individual member. For example, the questions (114) may include asking the healthcare provider whether the individual member has options for treating an adverse condition. The questions (114) can be prepared automatically through analysis of the communications (102).

The various sections of the record (104) described above may be provided in list form, such as a chronological listing of lifestyle activities engaged. Alternatively, the sections may be provided in a narrative form, such as, for example, detailing various relationships. The record (104) may also have more or less sections than described above.

The data repository (100) also stores expected interaction(s) (116). The expected interactions (116) are, for example, a time when an expected interaction between the individual member and their healthcare provider is expected to take place. The expected interaction (116) also includes a type of interaction (116) expected such as, for example, a new patient exam, a follow-up exam, an annual exam, etc. The expected interaction (116) can be predicted based on the communications (102).

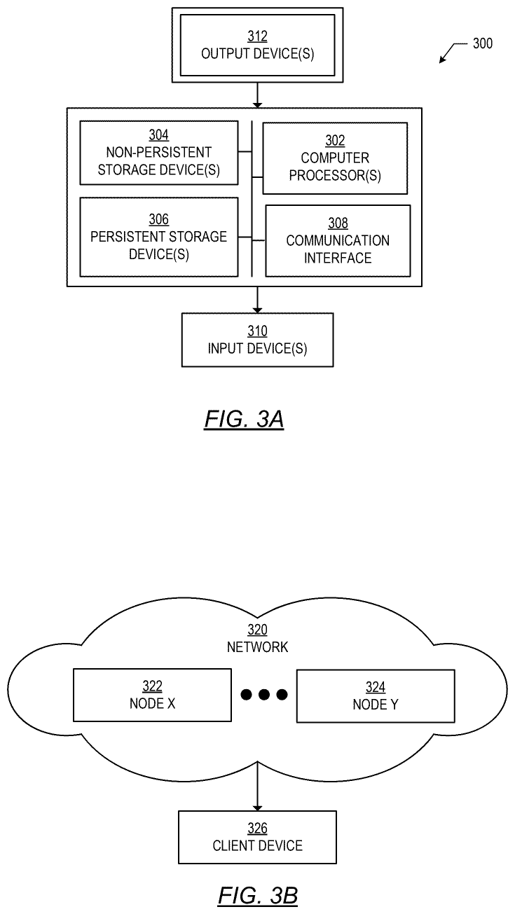

The system shown in FIG. 1 may include other components. For example, the system shown in FIG. 1 also may include a server (130). The server (130) is one or more computer processors, data repositories, communication devices, and supporting hardware and software. The server (130) may be in a distributed computing environment. The server (130) is configured to execute one or more applications, such as a communication analyzer (136). An example of a computer system and network that may form the server (130) is described with respect to FIG. 3A and FIG. 43.

The server (130) also includes a computer processor (132). The computer processor (132) is one or more hardware or virtual processors which may execute computer readable program code that defines one or more applications, such as the communication analyzer (136). An example of the computer processor (132) is described with respect to the computer processor(s) (302) of FIG. 3A.

The server (130) also may include a server controller (134). The server controller (134) is software or application specific hardware which, when executed by the computer processor (132), controls and coordinates operation of the software or application specific hardware described herein. Thus, the sever controller (134) may control and coordinate execution of the communication analyzer (136) and the language model (144).

The server (130) also includes a communication analyzer (136). The communication analyzer (136) is software or application specific hardware which, when executed by the computer processor (132), receives the communications (102) as input and can generate the record (104) based on the communications (102). The communication analyzer (136) may also generate a prompt for a language model (144), described in more detail below.

The server (130) also includes a digital communications network (DCN) (142). The DCN (142) is a network through which members of a population can interact with each other, or with a system supported by the DCN (142). The DCN (142) can also manage an individual member's communication feed to prioritize messages which contain ideas that might be applicable to them. This can be based on various factors, such as the individual member's own biological information, the individuals they follow in the network, etc.

The DCN (142) can also provide means for members of the population to communicate with each other. For example, the individual member can share their communications (102), their records (104), or any other communication with other members in the DCN (142). Communications between the members of the population in the DCN (142) can be, for example, one to one, one to many, one to the system, the system to one, or the system to many. Further, in some embodiments, the DCN (142) may use artificial intelligence (AI) to derive communications from the DCN (142) from an analysis of communications in the DCN (142) or from biometric data (108) and/or psychometric data (110) provided to the DCN (142) from the members.

The server (130) also includes a language model (144). The language model (144) is a natural language processing machine learning model. An example of the language model (144) may be a large language model, such as CHATGPT® or LLAMA®. However, many different language models may be used. The language model (144) may also be used to, for example, generate the record (104). For example, the language model (144) can receive a prompt from the communication analyzer (136) that includes the communications (102), desired sections of the record (104), a format for the record (104), a style for the record (104), etc.

The system shown in FIG. 1 also may include one or more user devices (150). The user devices (150) may be considered remote or local. A remote user device is a device operated by a third-party (e.g., an end user of a chatbot) that does not control or operate the system of FIG. 1. Similarly, the organization that controls the other elements of the system of FIG. 1 may not control or operate the remote user device. Thus, a remote user device may not be considered part of the system of FIG. 1.

In contrast, a local user device is a device operated under the control of the organization that controls the other components of the system of FIG. 1. Thus, a local user device may be considered part of the system of FIG. 1.

In any case, the user devices (150) are computing systems (e.g., the computing system (300) shown in FIG. 3A) that communicate with the server (130). The user devices (150) may include a wearable monitor (156) and be configured to send stress indicator data (110) to the server (130). In an alternative embodiment, a separate wearable device may be in communication with the user device (150), such as a smart watch, a HR monitor, a HRV monitor, a continuous glucose monitor, a continuous ketone monitor, a skin temperature monitor, and/or blood pressure monitor. The user devices (150) may also include a user input device (152) and/or a display device (154).

In contrast, a local user device is a device operated under the control of the organization that controls the other components of the system of FIG. 1. Thus, a local user device may be considered part of the system of FIG. 1.

While FIG. 1 shows a configuration of components, other configurations may be used without departing from the scope of one or more embodiments. For example, various components may be combined to create a single component. As another example, the functionality performed by a single component may be performed by two or more components.

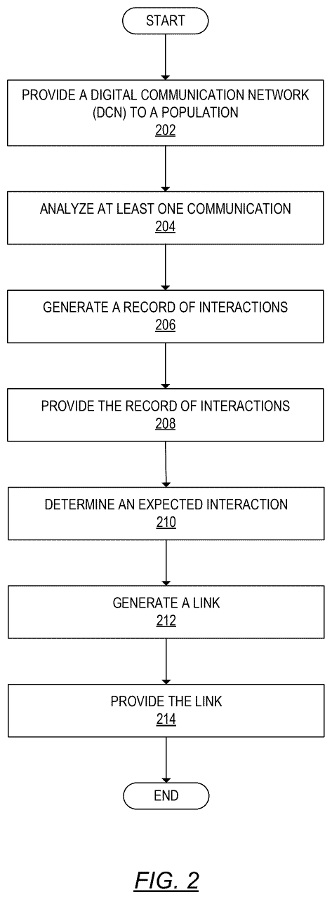

FIG. 2 is a logic flow diagram that illustrates a method, and a result of execution of computer program instructions, in accordance with various embodiments. The method can be used to improve communications between an individual member and a healthcare provider. The method can also, in other instances, be used in part to facilitate or accomplish at least one of: manage the health care cost of the population; reduce the health care risk in the population; and/or slow the progression of an adverse health condition.

At Block 202, a step of providing a digital communication network (DCN) to a population is provided. The DCN may be the same as or similar to the DCN (142). As previously described, members of the population enrolled in the DCN can communicate with each other or with the DCN itself.

At Block 204, a step of analyzing the at least one communication is provided. The communication may be the same as or similar to the communications (102) and may be analyzed by, for example, a communication analyzer such as the communication analyzer (136). The communication analyzer may, for example, analyze the communications to generate a record such as the record (104). The communication analyzer may also generate a prompt for a language model such as the language model (144) to generate the record.

At Block 206, a step of generating a record is provided. The record may be generated automatically by, for example, the communication analyzer, the language model, or an AI model based on the analysis of the communications. As previously described, the record can include one or more sections such as information about lifestyle activities such as the activities (106), biometric data such as the biometric data (108), psychometric data such as the psychometric data (110), relationship information such as the relationship information (112), and/or one or more questions such as the questions (114). As previously described, the various sections of the record can be used to assist the individual member during a meeting with their healthcare provider. For example, the questions may be used as prompts by the individual member to explore, for example, treatment options.

In some embodiments, the record can be used to gather information about the individual member and the individual member's health journey. In other words, the record can be used to aggregate relevant information regarding the individual member's health. The record can also be used to provide relevant information to, for example, the individual member's healthcare provider.

In still other embodiments, the record may catalogue situations that may be relevant to one or more adverse conditions that the individual member is experiencing. The adverse condition the individual member is experiencing can be determined by the analysis of the communications on the DCN. The adverse condition the individual member is experiencing may also be identified through dialog with a communication bot. One or more communications with the conversation bot may be used to prioritize the order in which the individual member sees communications on the DCN.

The record can also incorporate excerpts of communications on the DCN. The excerpts of communications may include post titles, post topics, quotes from the body of the communication, and/or post contributors. Also, the excerpts could be records of communications on the DCN. These records may be created automatically by the DCN and/or are based on a topic, a member's communications, or a time period.

The record can also recommend (and provides access through the DCN) tools to enhance the gathering of information on the adverse condition as part of preparation of the record. The tools could include a biosample, a wearable device, a psychometric instrument, a structured activity, etc.

The record created for the individual member by the DCN may also be designed to mimic the individual member's style. The record created for the individual member may alternatively be formulated in a style designed to provide a positive reaction in community members near the individual member in a social graph of the DCN.

The record created for the user may also be formulated in a style designed to provide a positive reaction in community members who have an adverse health condition associated with the communication. The health conditions could be any of: a chronic systemic inflammation, malaise or low energy, a disease (such as, Obesity, Diabetes, rheumatoid arthritis, Crohn's, Psoriasis, eczema, cardiovascular disease, congestive heart failure, chronic obstructive pulmonary disease, asthma, depression, anxiety, etc.), a health risk (such as, falling, becoming infected, etc.), a social dysfunction and/or a prodromal disease.

At Block 208, a step of providing the record to the individual member is provided. The record may be provided to the individual member via, for example, a user device such as the user device (150). The record can be provided when, for example, the individual member indicates directly or indirectly that they have an upcoming interaction with the healthcare system. Alternatively, the DCN may access the individual member's calendar to determine when the individual member wants a record or an updated record.

In some embodiments, the record may be provided to an influential member that may share their record or parts of their record with other members in the DCN. Such sharing may encourage other members to also obtain and/or use a record of their own health and/or health journey.

In other embodiments, the record may also be provided to the individual member as an aid or suggestion for preparing a post, message, or communication to the DCN. The suggestion may be prepared so that various details regarding the individual member are automatically removed such as, for example, private health information, sensitive data, etc.

At Block 210, a step of determining an expected interaction is provided. In some embodiments, the expected interaction may be determined by, for example, the DCN based on the analysis of communications by the individual member. For example, the communications may indicate that the individual member is going to visit their healthcare provider for an annual exam in one month. Thus, the DCN can automatically prepare the record for the individual member prior to the annual exam and tailor the record to information useful to the healthcare provider for an annual exam.

In some embodiments, the Block 208 of providing the record to the individual member may be provided prior to the Block 210 such that the individual member has the record to provide their healthcare provider. Further, the record may include the questions, which may be helpful for the individual member to have on hand to ask their healthcare provider.

At Block 212, a step of generating a link is provided. The link may be generated by, for example, the DCN and may link directly to the record.

At Block 214, a step of providing the link is provided. The link can be provided to the healthcare provider via the DCN. Such link can be provided so that the healthcare provider can, for example, view the record prior to meeting with the individual member.

The method described in FIG. 2 can include more or less steps. For example, the method of FIG. 2 may not include the steps of Blocks 210, 212, and/or 214. One or more steps or any combination of steps may also be repeated in the method described in FIG. 2.

The method described in FIG. 2 can be useful in many scenarios. For example, the records, when containing information about the health of the individual member, can be used to manage a variety of conditions such as, for example, chronic system inflammation (CSI). For example, specific inflammatory markers may be of interest, e.g., in diagnosing and approving drugs to treat rheumatoid arthritis (RA). Such markers may include, for example, C-reactive protein (CRP), which can be combined with other markers. Management of a condition can include maintaining the condition at the present levels, slowing the progression or reversing the condition (if possible). Using the DCN, many members of the population can be assisted which allows the embodiments to be used to manage the health of the population, the health risks of the population and/or reduce health care expense of the population overall.

By way of background, generalized CSI is not specific to any disease and, as such, does not often fit into a single category in a healthcare system. Such characterization results in CSI not being treated in a risk/benefit positive way with drugs or surgery or with any of the tools the provider and insurer community provide. Instead, it is driver and marker of disease progression in general, even if the particular disease is unknown. While the risk-benefit-ratio of drugs and surgery are not positive it can be improved (and therefore disease progression can be slowed) with lifestyle, social situation, emotional management with little or no risk.

Although CSI effects progression of a disease, progression is not typically measured in healthcare. Inflammation and its management impact disease progression and, at some point, disease state. If disease progression can be slowed, the disease state can be avoided and the population is less sick. Thus, it is beneficial to track CSI and whether an intervention has improved or not improved CSI through the individual member's communications and reflections in the DCN.

In another example, health care in a population can be improved by providing a means for members of said population to present improved health records via the records that can be automatically generated for said member. The communications in the DCN are analyzed to determine various factors for an individual member, which are then recorded in the individual member's record. The DCN can then send the record to the individual member or provide access to the record. In some cases, the process can be iterative, where the individual member can review the record and provide updates.

One or more embodiments may be implemented on a computing system specifically designed to achieve an improved technological result. When implemented in a computing system, the features and elements of the disclosure provide a significant technological advancement over computing systems that do not implement the features and elements of the disclosure. Any combination of mobile, desktop, server, router, switch, embedded device, or other types of hardware may be improved by including the features and elements described in the disclosure.

For example, as shown in FIG. 3A, the computing system (300) may include one or more computer processor(s) (302), non-persistent storage device(s) (304), persistent storage device(s) (306), a communication interface (308) (e.g., Bluetooth interface, infrared interface, network interface, optical interface, etc.), and numerous other elements and functionalities that implement the features and elements of the disclosure. The computer processor(s) (302) may be an integrated circuit for processing instructions. The computer processor(s) (302) may be one or more cores, or micro-cores, of a processor. The computer processor(s) (302) includes one or more processors. The computer processor(s) (302) may include a central processing unit (CPU), a graphics processing unit (GPU), a tensor processing unit (TPU), combinations thereof, etc.

The input device(s) (310) may include a touchscreen, keyboard, mouse, microphone, touchpad, electronic pen, or any other type of input device. The input device(s) (310) may receive inputs from a user that are responsive to data and messages presented by the output device(s) (312). The inputs may include text input, audio input, video input, etc., which may be processed and transmitted by the computing system (300) in accordance with one or more embodiments. The communication interface (308) may include an integrated circuit for connecting the computing system (300) to a network (not shown) (e.g., a local area network (LAN), a wide area network (WAN) such as the Internet, mobile network, or any other type of network) or to another device, such as another computing device, and combinations thereof.

Further, the output device(s) (312) may include a display device, a printer, external storage, or any other output device. One or more of the output device(s) (312) may be the same or different from the input device(s) (310). The input device(s) (310) and output device(s) (312) may be locally or remotely connected to the computer processor(s) (302). Many different types of computing systems exist, and the aforementioned input device(s) (310) and output device(s) (312) may take other forms. The output device(s) (312) may display data and messages that are transmitted and received by the computing system (300). The data and messages may include text, audio, video, etc., and include the data and messages described above in the other figures of the disclosure.

Software instructions in the form of computer readable program code to perform embodiments may be stored, in whole or in part, temporarily or permanently, on a non-transitory computer readable medium such as a solid-state drive (SSD), compact disk (CD), digital video disk (DVD), storage device, a diskette, a tape, flash memory, physical memory, or any other computer readable storage medium. Specifically, the software instructions may correspond to computer readable program code that, when executed by the computer processor(s) (302), is configured to perform one or more embodiments, which may include transmitting, receiving, presenting, and displaying data and messages described in the other figures of the disclosure.

The computing system (300) in FIG. 3A may be connected to, or be a part of, a network. For example, as shown in FIG. 3B, the network (320) may include multiple nodes (e.g., node X (322) and node Y (324), as well as extant intervening nodes between node X (322) and node Y (324)). Each node may correspond to a computing system, such as the computing system shown in FIG. 3A, or a group of nodes combined may correspond to the computing system shown in FIG. 3A. By way of an example, embodiments may be implemented on a node of a distributed system that is connected to other nodes. By way of another example, embodiments may be implemented on a distributed computing system having multiple nodes, where each portion may be located on a different node within the distributed computing system. Further, one or more elements of the aforementioned computing system (300) may be located at a remote location and connected to the other elements over a network.

The nodes (e.g., node X (322) and node Y (324)) in the network (320) may be configured to provide services for a client device (326). The services may include receiving requests and transmitting responses to the client device (326). For example, the nodes may be part of a cloud computing system. The client device (326) may be a computing system, such as the computing system shown in FIG. 3A. Further, the client device (326) may include or perform all or a portion of one or more embodiments.

The computing system of FIG. 3A may include functionality to present data (including raw data, processed data, and combinations thereof) such as results of comparisons and other processing. For example, presenting data may be accomplished through various presenting methods. Specifically, data may be presented by being displayed in a user interface, transmitted to a different computing system, and stored. The user interface may include a graphical user interface (GUI) that displays information on a display device. The GUI may include various GUI widgets that organize what data is shown, as well as how data is presented to a user. Furthermore, the GUI may present data directly to the user, e.g., data presented as actual data values through text, or rendered by the computing device into a visual representation of the data, such as through visualizing a data model.

Various operations described are purely exemplary and imply no particular order. Further, the operations can be used in any sequence when appropriate and can be partially used. With the above embodiments in mind, it should be understood that additional embodiments can employ various computer-implemented operations involving data transferred or stored in computer systems. These operations are those requiring physical manipulation of physical quantities. Usually, though not necessarily, these quantities take the form of electrical, magnetic, or optical signals capable of being stored, transferred, combined, compared, and otherwise manipulated.

Any of the operations described that form part of the presently disclosed embodiments may be useful machine operations. Various embodiments also relate to a device or an apparatus for performing these operations. The apparatus can be specially constructed for the required purpose, or the apparatus can be a general-purpose computer selectively activated or configured by a computer program stored in the computer. In particular, various general-purpose machines employing one or more processors coupled to one or more computer readable medium, described below, can be used with computer programs written in accordance with the teachings herein, or it may be more convenient to construct a more specialized apparatus to perform the required operations.

The procedures, processes, and/or modules described herein may be implemented in hardware, software, embodied as a computer-readable medium having program instructions, firmware, or a combination thereof. For example, the functions described herein may be performed by a processor executing program instructions out of a memory or other storage device.

The foregoing description has been directed to particular embodiments. However, other variations and modifications may be made to the described embodiments, with the attainment of some or all of their advantages. Modifications to the above-described systems and methods may be made without departing from the concepts disclosed herein. Accordingly, the invention should not be viewed as limited by the disclosed embodiments. Furthermore, various features of the described embodiments may be used without the corresponding use of other features. Thus, this description should be read as merely illustrative of various principles, and not in limitation of the invention.

What is claimed is:

1. A method for improving communications between an individual member and a healthcare provider, the method comprising:
providing a digital communication network (DCN) for a plurality of members of a population to communicate with each other;
analyzing at least one communication in the DCN;
generating a record for the individual member in the DCN based on the analysis of the at least one communication, the record including at least one of:
one or more lifestyle activities engaged in by the individual member,
biometric data reported to the DCN regarding the individual member, and
information regarding one or more relationships of the individual member; and
providing the record to the individual member through the DCN.

2. The method of claim 1, wherein the record comprises a section having one or more prepared questions the individual member can ask the healthcare provider, and wherein the one or more prepared questions include questions regarding medical care for the individual member.

3. The method of claim 2, wherein the questions are prepared automatically through the analysis of the at least one communication.

4. The method of claim 1, wherein the record is generated prior to an expected interaction with the healthcare provider by the individual member.

5. The method of claim 4, further comprising determining, by the DCN, the expected interaction based on the analysis of the at least one communication by the individual member.

6. The method of claim 1, wherein the record is prepared in at least one of a written format and a digital format.

7. The method of claim 1, further comprising:
generating a link to the record; and
providing the link to the healthcare provider.

8. The method of claim 1, wherein the lifestyle activities are prepared in at least one of a narrative form, a first person perspective, and a list form.

9. The method of claim 8, wherein the list form comprises at least one section of new activities, activities continued, activities dropped, and activities contemplated.

10. The method of claim 1, wherein the biometric data is derived in part from at least one of biometric data and psychometric data submitted to the DCN by the individual member.

11. The method of claim 10, wherein at least one of the biometric data and the psychometric data is derived from a biometric sample.

12. The method of claim 11, wherein the biometric sample is obtained from at least one of a home test, a wearable device, a psychometric instrument, a psychosocial instrument, and a psychological instrument.

13. The method of claim 12, wherein the wearable device comprises at least one of an accelerometer, a HR monitor, a HRV monitor, a continuous glucose monitor, a continuous ketone monitor, a skin temperature monitor.

14. The method of claim 11, wherein the biometric sample comprises at least one of a saliva sample, a blood sample, a breath sample and a stool sample.

15. The method of claim 12, further comprising providing the home test through the DCN.

16. The method of claim 1, wherein the record further includes analytics based on a combination of a plurality of biometric readings and changes in the biometric readings over time.

17. The method of claim 1, wherein the information regarding relationships is determined by analyzing at least one of contents of posts in the DCN and the at least one communications in the DCN.

18. The method of claim 1, wherein the method is used to at least one of: manage health care cost of the population; reduce health care risk in the population; and slow progression of an adverse health condition.

19. A method for improving communications between an individual member and a healthcare provider, the method comprising:
providing a digital communication network (DCN) for a plurality of members of a population to communicate with each other;
analyzing at least one communication in the DCN;
generating a record for the individual member in the DCN, the record including at least one of:
one or more lifestyle activities engaged in by the individual member,
biometric data reported to the DCN regarding the individual member,
information regarding one or more relationships of the individual member, and
one or more prepared questions the individual member can ask the healthcare provider;
determining an expected interaction between the individual member and the healthcare provided based on the analysis; and
providing the record prior to the expected interaction.

20. A system to improve communications between an individual member and a healthcare provider, the system comprising:
a computer processor;
a data repository in communication with the computer processor and storing:
at least one communication, and
a record having at least one of:
one or more lifestyle activities engaged in by the individual member,
biometric data reported to the DCN regarding the individual member, and
information regarding one or more relationships of the individual member
a communication analyzer which, when executed by the computer processor, analyzes the at least one communication;
a digital communications network which, when executed by the computer processor, provides a network for members of population to interact with each other; and
a server controller which, when executed by the computer processor:
generate the record for the individual member based on the analysis of the at least one communication; and
provide the record to the individual member.
